(19) United States
(12) Patent Application Publication
(10) Pub. No.: US 2011/0029076 A1
(43) Pub. Date: Feb. 3, 2011

(54) Breast Implant Therapeutic Delivery System

(76) Inventors: John D. Paletta, Pooler, GA (US); John P. Kennedy, Pooler, GA (US); Curtis E. Jones, Savannah, GA (US)

Correspondence Address: NIXON & VANDERHYE, PC, 901 NORTH GLEBE ROAD, 11TH FLOOR, ARLINGTON, VA, 22203, US

(21) Appl. No.: 12/847,356

(22) Filed: Jul. 30, 2010

Related U.S. Application Data
(60) Provisional application No. 61/229,781, filed on Jul. 30, 2009.

(51) Int. Cl. A61F 2/12

(52) U.S. Cl. 623/8

(57) ABSTRACT

A surgical procedure for breast augmentation includes the steps of preparing a pocket for receiving a breast implant; applying a semisolid including at least one therapeutic agent to at least one of the pocket or the breast implant; and inserting the breast implant in the pocket. An implant kit includes the implant and the semisolid with one or more additives.

### CROSS-REFERENCES TO RELATED APPLICATIONS

This application claims the benefit of U.S. Provisional Patent Application Ser. No. 61/229,781, filed Jul. 30, 2009, the entire content of which is herein incorporated by reference.

### STATEMENT REGARDING FEDERALLY SPONSORED RESEARCH OR DEVELOPMENT

(NOT APPLICABLE)

### BACKGROUND OF THE INVENTION

Breast implants and the implant surgical procedure have several well-known complications including pain, bleeding, bruising, infection, and capsular contracture. It would be desirable to minimize these conditions.

U.S. Pat. No. 6,488,952 describes a semisolid therapeutic delivery system and combination semisolid, multi-particulate, therapeutic delivery system. The semisolid material has been used to deliver chemotherapy for brain tumor patients. The contents of the '952 patent are hereby incorporated by reference.

### SUMMARY OF THE INVENTION

The semisolid is capable of being mixed with additives that can provide added functionality. It would be desirable for an implant procedure to incorporate the modified semisolid to reduce associated complications.

According to the described embodiments, compositions are disclosed with a number of advantages over previously known compositions in the field. The inventors have discovered that certain embodiments of liquid-crystal forming semisolids demonstrate advantageous utilities including: promoting hemostasis; promoting wound healing; providing barriers to seal tissues and preventing adhesions; promoting tissue growth; mimicking soft tissues; and inhibiting microbial infections including microbial adhesion upon medical devices and tissues. Furthermore, it has been discovered that: certain fatty acids, when added to certain formulations of liquid-crystal forming compounds, increase the viscosity or firmness of the liquid crystal phase once formed as well as extend their microbial spectrum for microbial adhesion inhibition; the compositions and formulations provide excellent toxicity, sensitization and irritation profiles; the compositions may be designed to be biodegradable; all of which offer advantages for their utility in the surgical procedures disclosed in the embodiments herein.

In an exemplary embodiment, a surgical procedure for breast augmentation includes the steps of preparing a pocket for receiving a breast implant; applying a semisolid including at least one therapeutic agent to at least one of the pockets or the breast implant; and inserting the breast implant in the pocket. The method may further include, prior to the applying step, the step of incorporating an additive into the semisolid, where the additive is effective for minimizing at least one of pain, bleeding, bruising, infection, and capsular contracture.

In second exemplary embodiment, a surgical procedure for breast augmentation includes the steps of preparing a pocket for receiving a breast implant; inserting the breast implant in the pocket; and implanting a semisolid including at least one therapeutic agent within the pocket. The method may further include, prior to the semisolid implantation, the step of incorporating an additive into the semisolid, where the additive is effective for minimizing at least one of pain, bleeding, bruising, infection, and capsular contracture.

In one embodiment, the additive is a local anesthetic. In this context, the method may include the step of controlling a duration of anesthetic delivery. Alternatively or additionally, the additive may be an antimicrobial agent.

The applying step may be practiced by instilling the semisolid into the pocket prior to inserting the breast implant. Alternatively, the applying step may be practiced by applying the semisolid directly to the implant prior to inserting the breast implant. As a another alternative, the applying step may be practiced by implanting the semisolid into the pocket after inserting the breast implant.

In another exemplary embodiment, a breast implant kit includes at least one breast implant and a packaged semisolid including at least one therapeutic agent and an additive, where the additive is effective for minimizing at least one of pain, bleeding, bruising, infection, and capsular contracture. The kit may include a plurality of additives separate from the semisolid, where the additives have different purposes and effects, and where the additives are selectively mixable with the semisolid prior to use.

### DETAILED DESCRIPTION OF THE INVENTION

In a typical breast augmentation procedure, after making access incisions, a dissection is performed to the appropriate plane, e.g., sub-glandular (under the breast) or sub-muscular (under the muscle) or subcutaneous (under the skin). A pocket is developed by separating the area from the underlying layer. Hemostasis is achieved (i.e., bleeding is controlled), and the pocket is irrigated with saline or an antibiotic solution. The implant is placed in the pocket, and if a saline implant is being used, the implant is inflated. Sometimes a “sizer” is used prior to the permanent implant. After placing the implant, the wound is closed. Some surgeons apply a local anesthetic in the pocket as they are closing the wound.

In preferred embodiments, the method of implantation incorporates the semisolid with an additive directly on the implant. That is, the semisolid is placed directly on the implant prior to placement in the pocket. The semisolid may be packaged together with or separately from the implant. In an alternative procedure, the semisolid may be instilled into the pocket just prior to placing the implant. Still further, the semisolid may be implanted between the breast implant and patient tissues after insertion of the breast implant with a suitable syringe or the like.

In one embodiment, there is provided a therapeutic formulation effective for controlling pain, bleeding, bruising, infection, and/or capsular contracture, at a desired implant site in a subject, the formulation comprising about 60% to about 97% by weight liquid-crystal forming compound and 0% to about 40% by weight solvent. In related embodiments, the solvent may be a polar solvent, a non-polar solvent, a semi-polar solvent or a combination thereof, and particular embodiments may comprise about 95% liquid-crystal forming compound and about 5% normal saline solution, purified water, ethanol 190 proof or mixtures of any two; 90% liquid-crystal forming compound and about 10% normal saline solution, purified water, ethanol 190 proof or mixtures of any two; 88% liquid-crystal forming compound and about 12% normal saline solution, purified water, ethanol 190 proof or mixtures of any two; 85% liquid-crystal forming compound and about 15% normal saline solution, purified water, ethanol 190 proof or mixtures of any two; or 75% liquid-crystal forming compound and about 25% normal saline solution, purified water, ethanol 190 proof or mixtures of any two.

In other embodiments, the composition effective for controlling pain, bleeding, bruising, infection, and/or capsular contracture at a desired implant site in a subject, provides utility as an anti-adherent between the implant and bodily tissue to assist in placement or removal of the implant from a site of use thereby reducing trauma from application or removal of the implant, and the biological fluid controlling formulation may be applied to the implant by spray coating, hot-melt coating, dip coating, direct transfer, manual application or a combination thereof. Specific embodiments provide an implant that may be any of a breast implant, tissue spacer, tissue expander, tissue filler or hardware, and the liquid-crystal forming compound may be any of a fatty acid ester, a polyethylene oxide, a glycolipid, a polyester, a polyethylene glycol, or a combination thereof. In related embodiments, the fatty acid ester may be a monoester, diester, triester or mixture thereof, and the monoester may be the group of glyceryl monoarachidonate, glyceryl monolinoleate, glyceryl monolinolenate, glyceryl monopalmitoleate, glyceryl monooleate, glyceryl monoerucate, isopropyl monoarachidonate, isopropyl monolinoleate, isopropyl monolinolenate, isopropyl monopalmitoleate, isopropyl monooleate, methyl monoarachidonate, methyl monolinoleate, methyl monolinolenate, methyl monopalmitoleate, methyl monooleate, propylene glycyl monoarachidonate, propylene glycyl monolinoleate, propylene glycyl monolinolenate, propylene glycyl monopalmitoleate, propylene glycyl monooleate, and a combination thereof. For applications that optimally require highly viscous liquid crystalline states to be formed, fatty acid esters, phospholipids and glycolipids include the liquid-crystal forming compound that are preferable alone or in combination with others, glycerol monooleate, glycerol monoerucate, phosphatidylcholine and phosphatidylethanolamine providing more cost effective examples for applications requiring bulk quantities. In related embodiments, the solvent may be any of an alcohol, polyethylene glycol, propylene glycol, polypropylene glycol, water, isotonic aqueous solution, a physiologic buffered system, or combination thereof; the liquid-crystal forming compound may be any of a fatty acid monoester, fatty acid diester, fatty acid triester or combination thereof further comprising at least one unsaturated carbon-carbon bond. More particularly, the liquid crystal forming-agent may be a glyceryl monoester, diester, triester, or combination thereof, and still more particularly, the liquid-crystal forming compound may be glyceryl monooleate or glyceryl monoerucate.

### EXAMPLES

### Example 1

| | |
|---|---|
| Purified Water, USP | 5% |
| Glyceryl monooleate | 95% |

### Example 2

| | |
|---|---|
| Normal Saline for Injection, USP | 5% |
| Glyceryl monooleate | 95% |

### Example 3

| | |
|---|---|
| Ethanol, 190 proof | 5% |
| Glyceryl monooleate | 95% |

Purified water, USP, Normal Saline or Ethanol was heated to approximately 400 C. Glyceryl Monooleate (GMO) was heated to melting. The solvent was combined with GMO. The resulting system was well mixed and allowed to return to ambient temperature undisturbed. The resulting mixture produced a hazy liquid formulation with a viscosity in the approximate range of 80-500 centipoise.

The present example possessed characteristics making it operable for controlling pain, bleeding, bruising, infection, and/or capsular contracture at a desired implant site in a subject. The formulation may also be used in conjunction with device implants to inhibit microbial adhesion, inhibit capsular contraction, inhibit inflammatory reaction, inhibit tissue adhesion and, with addition of anesthetic agents control pain locally.

### Example 4A

| | |
|---|---|
| Ethanol, 190 proof | 1% |
| Purified Water, USP | 11% |
| Glyceryl monooleate | 88% |

### Example 4B

| | |
|---|---|
| Ethanol, USP | 2% |
| Normal Saline for Injection, USP | 10% |
| Monoerucin | 88% |

In both examples, ethanol and normal saline or purified water was mixed thoroughly and heated to approximately 40° C. in separate closed containers. Glyceryl Monooleate (GMO) or Monoerucin were heated to melting in separate containers. The corresponding solvent mixtures were combined with GMO and Monoerucin respectively. The resulting systems were well mixed and allowed to return to ambient temperature undisturbed. The resulting mixtures produced hazy liquid formulations with a viscosity in the approximate range of 80-500 centipoise.

The present examples possessed characteristics making them operable for controlling pain, bleeding, bruising, infection, and/or capsular contracture at a desired implant site in a subject. The formulations may also be used in conjunction with device implants to inhibit microbial adhesion, inhibit capsular contraction, inhibit inflammatory reaction, inhibit tissue adhesion and, with addition of anesthetic agents control pain locally.

### Example 5

| | |
|---|---|
| Polyethylene Glycol (PEG) 400, NF | 10% |
| Polyethylene Glycol (PEG) 200, NF | 5% |
| Glyceryl monooleate | 85% |

PEG 400, NF and PEG 200, NF were mixed and heated to approximately 400 C. Glyceryl Monooleate (GMO) was heated to melting. The PEG mixture was combined with GMO. The resulting system was well mixed and allowed to return to ambient temperature undisturbed. The resulting mixture produced a clear liquid formulation with a viscosity in the approximate range of 80-200 centipoise. In the present embodiment, other MW PEGs may be useful as well and interchanged with those described above to produce alternative formulations having similar properties making such formulations operable for hemostatic applications.

The present examples possessed characteristics making them operable for controlling pain, bleeding, bruising, infection, and/or capsular contracture at a desired implant site in a subject. The formulations may also be used in conjunction with device implants to inhibit microbial adhesion, inhibit capsular contraction, inhibit inflammatory reaction, inhibit tissue adhesion and, with addition of anesthetic agents control pain locally.

### Example 6

| | |
|---|---|
| Sodium Hyaluronate | 2.5% |
| Purified Water, USP | 5% |
| Glyceryl monooleate | 92.5% |

The Sodium Hyaluronate was dissolved in the purified water and heated to approximately 350 C. Glyceryl Monooleate (GMO) was heated to melting. The Sodium Hyaluronate solution was combined with GMO. The resulting system was well mixed and allowed to return to ambient temperature undisturbed. The resulting mixture produced a hazy liquid formulation with a viscosity in the approximate range of 1000-3000 centipoise.

The present examples possessed characteristics making them operable for controlling pain, bleeding, bruising, infection, and/or capsular contracture at a desired implant site in a subject. The formulations may also be used in conjunction with device implants to inhibit microbial adhesion, inhibit capsular contraction, inhibit inflammatory reaction, inhibit tissue adhesion and, with addition of anesthetic agents control pain locally.

### Example 7

| | |
|---|---|
| Glyceryl monooleate | 86.4% |
| Lauric Acid | 0.9% |
| Capric Acid | 0.7% |
| Ethanol, USP | 1.4% |
| Purified Water, USP | 10.6% |

In a 14-day bacteriocidal challenge per Antimicrobial Effectiveness Test, the above composition was inoculated with S. Aureus at 1.3×105 CFU/g, E. Coli at 1.5×105 CFU/g and P. Aeruginosa at 2.7×105 CFU/g. All bacteria were dead at the end of the 14 day period. The above composition was also tested for the ability to inhibit microbial adhesion in a multispecies in vitro model. Concentrations as low as 0.1% were able to prevent microbial adhesion. Concentrations as low as 0.005% still had significant activity against microbial adhesion.

### Example 8

| | |
|---|---|
| Monoerucin | 85.1% |
| Lauric Acid | 1.1% |
| Capric Acid | 0.8% |
| Ethanol, USP | 1.7% |
| Normal Saline for Injection, USP | 11.3% |

In a 14-day bacteriocidal challenge per Antimicrobial Effectiveness Test, the above composition was inoculated with S. Aureus at 1.3×105 CFU/g, E. Coli at 1.5×105 CFU/g and P. Aeruginosa at 2.7×105 CFU/g. All bacteria were dead at the end of the 14 day period. The above composition was also tested for the ability to inhibit microbial adhesion in a multispecies in vitro model. Concentrations as low as 0.1% were able to prevent microbial adhesion. Concentrations as low as 0.005% still had significant activity against microbial adhesion.

### Example 9

| | |
|---|---|
| Glyceryl monooleate | 81.4% |
| Lauric Acid | 0.9% |
| Capric Acid | 0.7 |
| Lidocaine HCl | 5.0% |
| Ethanol, USP | 1.5% |
| Purified Water, USP | 10.5% |

### Example 10

| | |
|---|---|
| Glyceryl monooleate | 76.4% |
| Lauric Acid | 0.9% |
| Capric Acid | 0.7 |
| Lidocaine HCl | 10.0% |
| Ethanol, USP | 1.5% |
| Purified Water, USP | 10.5% |

### Example 11

| | |
|---|---|
| Glyceryl monooleate | 84.4% |
| Lauric Acid | 0.9% |
| Capric Acid | 0.7 |
| Bupivacaine HCl or Levobupivacaine | 2.0% |
| Ethanol, USP | 1.5% |
| Purified Water, USP | 10.5% |

### Example 12

| | |
|---|---|
| Glyceryl monooleate | 81.4% |
| Lauric Acid | 0.9% |
| Capric Acid | 0.7 |
| Tetracaine HCL | 5% |
| Ethanol, USP | 1.5% |
| Purified Water, USP | 10.5% |

### Example 13

| | |
|---|---|
| Glyceryl monooleate | 78.9% |
| Lauric Acid | 0.9% |
| Capric Acid | 0.7 |
| Tetracaine: Lidocaine (equal mix) | 7.5% |
| Ethanol, USP | 1.5% |
| Purified Water, USP | 10.5% |

The above composition was tested for the ability to inhibit microbial adhesion in a multi-species in vitro model. Concentrations as low as 0.1% prevented the formation of microbial adhesion. Concentrations as low as 0.005% still had significant activity against microbial adhesion. The present example possessed characteristics making it operable for controlling pain, bleeding, bruising, infection, and/or capsular contracture at a desired implant site in a subject. The formulation may also be used in conjunction with device implants to inhibit microbial adhesion, inhibit capsular contraction, inhibit inflammatory reaction, inhibit tissue adhesion and, with addition of anesthetic agents control pain locally.

In any disclosed formulation, the augmentative agent or therapeutic may be suspended in the formulation, dissolved in the formulation or a combination thereof.

Additive agents including antimicrobials, antibiofilm agents, anesthetics and sympathomimetics, may be added or mixed into the semisolids disclosed herein via dissolving into the base semisolid, suspended within the base semisolid or combinations thereof. With the addition of local anesthetic (lidocaine, marcaine, etc.) to the semisolid, a consistent amount of anesthetic can be delivered within the pocket for an extended period of time. The duration of drug delivery can be modified to provide pain control from 1 day to up to two weeks.

Additionally, the development of capsular contracture is known to be reduced by antimicrobial agents, purportedly related to reduction of the inflammatory response to low level microbial content introduced during the procedure. The use of the semisolid will limit microbial attachment to the implant surfaces as well as propagation, thereby reducing the rate of capsular contracture. Additionally, the semisolid has some mild hemostatic properties that will decrease bruising, inflammation and pain.

An antimicrobial agent can be added to decrease the risk of acute infection, but this may be decreased with the semisolid alone. If a particular facility/surgeon has a high rate of infection, then the causative agent can be identified, and a custom semisolid with appropriate antimicrobial or other additive can be made without having to change the technique.

The semisolid will be tested against the implant to assess any effects on implant and semisolid, particularly with regard to any effect on the structural integrity of the implant.

This technology can have implication for all implants (mesh used for hernia repair, orthopedic implants, infusion ports, PICC lines and central lines, vascular grafts, etc.).

Additionally, the semisolid may be effective to replace the currently used pain pumps. Pain pumps have a balloon-like reservoir that pushes local anesthetic into a small catheter with multiple openings, allowing the fluid to drip into the surgical site (similar to a soaker hose). Typical flow rates are 2-5 cc/hr. Pain pumps are expensive, and they are prone to malfunction such as the catheter being dislodged, kinked or cracked. Pain pumps also take additional surgeon and staff time to prepare and place. The use of the semisolid described herein can have a significant impact in post-operative pain control, not just implant cases. The semisolid will be easier to administer, without the malfunction risk and is a less expensive alternative. It is envisioned that the semisolid may have applications in a multitude of surgical procedures (tummy tuck, breast reduction, breast biopsy, hernia repair, fracture repair, hysterectomy, vasectomy, etc.).

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A surgical procedure for breast augmentation, comprising in any order:
(a) preparing a pocket for receiving a breast implant;
(b) inserting the breast implant in the pocket; and
(c) applying a semisolid including at least one therapeutic agent to the outer surface of the breast implant.

2. The surgical procedure according to claim 1, further comprising (d) incorporating an additive into the semisolid, wherein the additive is effective for minimizing at least one of pain, bleeding, bruising, infection, and capsular contracture.

3. The surgical procedure according to claim 2, wherein the additive comprises a local anesthetic.

4. The surgical procedure according to claim 3, further comprising controlling a duration of anesthetic delivery.

5. The surgical procedure according to claim 2, wherein the additive comprises an antimicrobial agent.

6. The surgical procedure according to claim 1, wherein the applying step is practiced by instilling the semisolid into the pocket prior to inserting the breast implant.

7. The surgical procedure according to claim 1, wherein the applying step is practiced by applying the semisolid directly to the implant prior to inserting the breast implant.

8. A breast implant kit comprising:
at least one breast implant; and
a packaged semisolid including at least one therapeutic agent and an additive, the additive being effective for minimizing at least one of pain, bleeding, bruising, infection, and capsular contracture.

9. The breast implant kit according to claim 8, comprising a plurality of additives separate from the semisolid, the additives having different purposes and effects, wherein the additives are selectively mixable with the semisolid prior to use.

10. The breast implant kit according to claim 8, wherein the additive comprises a local anesthetic.

11. The breast implant kit according to claim 8, wherein the additive comprises an antimicrobial agent

12. The breast implant kit according to claims 8, wherein the semisolid comprises:
about 60% to about 97% by weight liquid-crystal forming compound; and
0% to about 40% by weight solvent.
